(19) United States
(12) Patent Application Publication
(10) Pub. No.: US 2016/0209423 A1
(43) Pub. Date: Jul. 21, 2016

(54) METHODS FOR PREDICTING, DIAGNOSING OR MONITORING INFECTIONS OR CONDITIONS

(71) Applicant: UNIVESITY COLLEGE DUBLIN, NATIONAL UNIVERSITY OF IRELAND, DUBLIN, Dublin (IE)

(72) Inventors: Pauline Rudd, County Wicklow (IE); Henning Stöckmann, Dublin (IE); Stephen Carrington, County Wicklow (IE); Erin Williams, Dublin (IE); Tharmala Tharmalingam, Thousand Oaks, CA (US); Alexander Evans, County Donegal (IE)

(73) Assignee: UNIVESITY COLLEGE DUBLIN, NATIONAL UNIVERSITY OF IRELAND, DUBLIN, Dublin (IE)

(21) Appl. No.: 14/915,969

(22) PCT Filed: Sep. 3, 2014
(86) PCT No.: PCT/EP2014/068734

§ 371 Date: Mar. 2, 2016

(30) Foreign Application Priority Data
Sep. 4, 2013 (GB) 1315705.2

(51) Int. Cl. G01N 33/68

(52) U.S. Cl. CPC G01N 33/6854

(57) ABSTRACT

Provided is a method for predicting, diagnosing or monitoring an infection or condition caused by or associated with one or more microorganisms in a non-human female animal subject, comprising generating a glycosylation profile from a glycan-containing test sample of the subject, determining from the glycosylation profile one or more test values of one or more glycosylation markers of the infection or condition, comparing the one or more test values of the one or more glycosylation markers to one or more threshold values of the one or more glycosylation markers, and providing a prediction or diagnosis of the infection or condition based on the comparison.

This invention relates to methods for predicting, diagnosing or monitoring infections or conditions, in particular postpartum infections or conditions in animals, including infections or conditions of the uterus and mammary glands.

Postpartum infections or conditions such as endometritis, metritis and mastitis are known to cause substantial problems within the agricultural industry, affecting animals including cows and horses. For example, conditions involving uterine infection e.g. endometritis or metritis in postpartum cows is a significant cause of ill health in cattle and results in substantial economic losses within the agricultural sector. Almost all cows suffer uterine bacterial contamination after calving, and while some animals clear this, infection persists in others. Estimates of postpartum uterine infection range between 20% and a staggering 75%, and the incidence has been increasing in recent years, possibly due to increasing production pressure on cows. Uterine infection causes a marked decrease in fertility: calving to conception intervals are extended by 19 days and conception rate to first service is reduced by 20%. Clearance of infection is required for the establishment of pregnancy but conception rates remain 20% lower, and 3% of affected cows remain infertile even after clinical resolution of disease.

At present, clinical diagnosis of uterine disease typically occurs 2-5 weeks after calving, by which time substantial damage to health, productivity and long term fertility has been done. Losses due to uterine infection are estimated at [symbol] 292/cow/year. Thus, with over 1 million dairy cows in the national herd in Ireland, uterine disease costs Ireland between [symbol] 54- [symbol] 219 million every year, with costs of [symbol] 1.4-5.2 billion estimated for the EU and [symbol] 15-58 billion estimated for the global dairy industry.

The definitive diagnosis of uterine disease is made following histological examination of uterine biopsies to identify endometrial pathology. Analysis of bacteria isolated from uterine swabs may also be valuable as a diagnostic tool as specific bacteria, such as Escherichia coli and Trueperella pyogenes, have been shown to be associated with uterine disease. In the field, however, diagnosis of uterine disease usually relies on clinical examination. Clinical assessment of vaginal mucus is widely used to diagnose uterine infection as it has been shown to correlate with the number and type of bacteria in the uterus. Transrectal palpation can be used to identify animals with delayed involution of the uterus and/or cervix, which is associated with uterine infection. This is more accurately assessed using ultrasonography to enable visualisation of the reproductive tract. Fluid in the uterus can also be identified via ultrasound as it appears black and non-echogenic. Any purulent material is visible as grey flecks.

There are disadvantages associated with the known methods of diagnosis. For example, histological and bacteriological examination of uterine tissue biopsies, swabs or cytological brushings, are costly and time consuming. They require specific expertise and sample processing, and are not clinically feasible in most situations. These methods may even have negative effects on subsequent fertility. In particular, biopsies, because of their invasive nature, may traumatise tissues and potentiate infection.

Clinical examination of vaginal mucus is quick and cheap. However, not all cases of uterine disease are associated with purulent mucus discharge and mucus assessment will not identify these cases. Furthermore, it is unclear whether mucus in the vagina indicates the condition endometritis or a local vaginal infection, and an incorrect diagnosis will adversely affect the targeting of treatment. Transrectal palpation to identify delayed uterine involution is a generally ineffective technique for evaluating uterine infection because the rate and extent of uterine involution varies between cows. Ultrasonography improves diagnosis when used in conjunction with other tests. However, used alone it is only able to detect severe cases of disease.

In summary, the existing technologies used to diagnose uterine disease in cattle are costly, labour intensive and invasive. Furthermore, the infection or condition is usually only diagnosed once it is well established. At this point, substantial damage to the cow's health, productivity and fertility has already been done.

The diagnosis of mastitis is most commonly achieved by examining a milk sample for blood or clotting, with subclinical cases being identified through somatic cell counts. However, the identification of cows that are predisposed to mastitis is less certain. Better indicators of such predisposition would be of benefit to the management of dairy enterprises because it could lead to early interventions capable of reducing economic losses associated with mastitis and including milk withdrawal, subfertility, and reduced milk quality (which is determined in part through somatic cell counts).

Accordingly, it is an object of the present invention to mitigate the disadvantages of the prior art methods.

It is also an object of the present invention to provide a novel method which is simple, accurate and inexpensive and which may not only be used to diagnose postpartum infections and conditions, but may also, importantly, be used to predict postpartum infections or conditions.

According to the invention there is provided a method for predicting, diagnosing or monitoring an infection or condition caused by or associated with one or more microorganisms in a non-human female animal subject, comprising

generating a glycosylation profile from a glycan-containing test sample of the subject,

determining from the glycosylation profile one or more test values of one or more glycosylation markers of the infection or condition,

comparing the one or more test values of the one or more glycosylation markers to one or more threshold values of the one or more glycosylation markers, and

providing a prediction or diagnosis of the infection or condition based on the comparison.

As used herein, the term “glycan” is intended to mean a monosaccharide, oligosaccharide or polysaccharide. The glycan may be part of, or derived from, a glycoprotein. Optionally, the glycan is a N-linked glycan, wherein the term “N-linked glycan”, synonymously known as a “N-glycan”, means that the glycan is attached to the nitrogen in the side chain of asparagine when it is part of a glycoprotein.

As used herein, the term “glycosylation profile” is intended to mean a presentation of one or more glycan structures present in a test sample. A glycosylation profile may be presented, for example, as a plurality of peaks each corresponding to one or more glycan structures present in the test sample. The step of generating a glycosylation profile from a glycan-containing test sample may be carried out using any suitable method. For example, without intending to limit the invention thereto, the step of generating the glycosylation profile may be carried out using liquid chromatography, optionally hydrophilic interaction liquid chromatography and/or ultra performance liquid chromatography as described herein, or using capillary electrophoresis (CE) or using mass spectrometry, or a combination thereof. Mass spectrometry may comprise using a time of flight (TOF) analyser or quadrupole mass filters or ion traps or orbitraps or Fourier transform ion cyclotron resonance, or a combination thereof. Glycan analysis methods that are suitable for generating a glycosylation profile are described in Marino et al. (2010) A systematic approach to protein glycosylation analysis: a path through the maze. Nat. Chem Biol. 6: 713-723.

As used herein, the term “providing a prediction or diagnosis of the infection or condition” is intended to mean being able to classify whether the subject is likely or unlikely to develop the infection or condition, or whether the subject has or does not have the infection or condition, as appropriate.

Optionally, the one or more test values of the one or more glycosylation markers comprises one or more levels of fucosylation of one or more glycans present in the test sample.

Optionally, the one or more test values of the one or more glycosylation markers comprises the degree of fucosylation of one or more glycans present in the test sample.

Optionally, the degree of fucosylation is calculated by dividing the peak area of a fucosylated form of a glycan present in the test sample, by the sum of the peak areas of the fucosylated and afucosylated forms of the glycan. Advantageously, the degree of fucosylation may be determined from the glycosylation profile.

As used herein, the term “fucosylation” is intended to mean a type of glycosylation involving fucose, in particular the attachment of a fucose residue to N-glycans. As used herein, the term “fucose” is intended to mean deoxy-galactose, where galactose has lost one hydroxyl group (—OH).

In some embodiments, if the one or more test values of the one or more glycosylation markers is above the one or more threshold values of the one or more glycosylation markers, the one or more test values is indicative of a prediction or diagnosis of the infection or condition.

In some embodiments, if the one or more test values of the one or more glycosylation markers is below the one or more threshold values of the one or more glycosylation markers, the one or more test values is indicative of a prediction or diagnosis of the infection or condition.

Optionally, a degree of fucosylation of one or more glycans in the test sample above a threshold value of the degree of fucosylation, is indicative of a prediction or diagnosis of the infection or condition.

Optionally, the one or more glycans is selected from the glycans A2G0, A2[3]G1, A2[6]G1 and A2G2, or a combination thereof.

Optionally, a degree of fucosylation of the glycan A2[6]G1 present in a prepartum test sample, above a threshold value of about 72%, is indicative of a prediction of the infection or condition.

Optionally, a degree of fucosylation of the glycan A2[6]G1 present in a day 7 test sample, above a threshold value of about 75%, is indicative of a prediction or diagnosis of the infection or condition.

Optionally, a degree of fucosylation of the glycan A2[6]G1 present in a day 14 test sample, above a threshold value of about 75%, is indicative of a prediction or diagnosis of the infection or condition.

Optionally, a degree of fucosylation of the glycan A2G2 present in a prepartum test sample, above a 30 threshold value of about 86%, is indicative of a prediction of the infection or condition.

Optionally, a degree of fucosylation of the glycan A2G2 present in a day 14 test sample, above a threshold value of about 90%, is indicative of a prediction or diagnosis of the infection or condition.

Optionally, a degree of fucosylation of the glycan A2G0 present in a day 14 test sample, above a threshold value of about 81%, is indicative of a prediction or diagnosis of the infection or condition.

Optionally, a degree of fucosylation of the glycan A2[3]G1 present in a day 14 test sample, above a threshold value of about 87%, is indicative of a prediction or diagnosis of the infection or condition.

Optionally, a degree of fucosylation of a combination of the glycans A2G0, A2[3]G1, A2[6]G1 and A2G2 present in a day 14 test sample, above a threshold value of about 85%, is indicative of a prediction or diagnosis of the infection or condition.

It will be appreciated that the degree of fucosylation is dependent on the glycan(s) investigated. The degree of fucosylation is also dependent on the day that the sample is taken (e.g. prepartum versus day 7 versus day 14).

Optionally, the one or more test values of the one or more glycosylation markers comprises the degree of contribution of one or more glycans present in the test sample to the total glycan pool.

Optionally, the degree of contribution of one or more glycans present in the test sample to the total glycan pool is calculated by dividing the peak area of a glycan, by the sum of the total peak area of the total glycans. Advantageously, the degree of contribution of one or more glycans present in the test sample to the total glycan pool may be determined from the glycosylation profile.

Optionally, a degree of contribution of one or more glycans in the test sample below a threshold value of the degree of contribution, is indicative of a prediction or diagnosis of the infection or condition.

Optionally, a degree of contribution of peak 10 present in a day 14 test sample, below a threshold value of about 3.5%, is indicative of a prediction or diagnosis of the infection or condition. As will be apparent from Table 1, peak 10 represents the glycan A2[3]G1.

It will be appreciated that the degree of contribution of the one or more glycans/peaks is dependent on the glycan(s)/peak(s) investigated. The degree of contribution of the one or more glycans/peaks is also dependent on the day that the sample is taken (e.g. prepartum versus day 7 versus day 14).

As used herein, the term “prepartum” test sample is intended to mean a test sample taken from the subject from about 25 days prepartum to about 1 day prepartum, optionally from about 10 days prepartum to about 1 day prepartum, further optionally about 7 days or 6 days or 5 days or 4 days or 3 days or 2 days or 1 day prepartum.

As used herein, the term “day 7” test sample is intended to mean a test sample taken from the subject from about day 5 postpartum to about day 11 postpartum, optionally from about day 5 postpartum to about day 9 postpartum, further optionally day 6 or day 7 or day 8 postpartum, still further optionally day 7 postpartum.

As used herein, the term “day 14” test sample is intended to mean a test sample taken from the subject from about day 12 postpartum to about day 18 postpartum, optionally from about day 12 postpartum to about day 16 postpartum, further optionally day 13 or day 14 or day 15 postpartum, still further optionally day 14 postpartum.

Suitable non-human female animal subjects include, but are not limited to, ruminants and non-ruminants.

Ruminants include, but are not limited to, cows, sheep, goats or camels, optionally cows.

Non-ruminants include, but are not limited to, horses or pigs.

Optionally, the subject is selected from a cow or a horse, further optionally a cow.

As used herein, the term “infection or condition caused by or associated with one or more microorganisms” is intended to include infections and conditions which are directly caused by one or more microorganisms and infections and conditions which may have some association with another factor, e.g. a compromised immune system, but which still are associated with one or more microorganisms to some degree. Suitable microorganisms include bacteria, protozoa, fungi, and viruses, optionally, bacteria and/or viruses.

Suitable infections and conditions include, but are not limited to, inflammatory conditions. Suitable infections and conditions also include, but are not limited to, infections or conditions of the female reproductive tract or a part thereof, optionally the uterus, or infections or conditions of the mammary gland, or a combination thereof. Suitable infections or conditions include postpartum infections or conditions. Suitable infections or conditions of the reproductive tract or a part thereof include, but are not limited to, vulvitis, vaginititis, cervicitis, metritis and endometritis, optionally metritis or endometritis, further optionally endometritis. Suitable infections or conditions of the mammary gland include mastitis. It will be appreciated that more than one infection or condition may be predicted and/or diagnosed in a subject.

Protozoa capable of causing, or which are associated with, infections or conditions of the reproductive tract include, but are not limited to, Tritrichomonas foetus and Topxoplasma gondii. Bacteria capable of causing, or which are associated with, metritis and endometritis, include, but are not limited to, Escherichia coli, Trueperella pyogenes, Prevotella species and Fusobacterium necrophorum. Viruses capable of causing, or which are associated with, metritis and endometritis, include, but are not limited to, Bovine Herpesvirus 4 (BoHV-4). Bacteria capable of causing, or which are associated with, mastitis, include, but are not limited to, Pseudomonas aeruginosa, Staphylococcus aureus, Staphylococcus epidermidis, Streptococcus agalactiae, Streptococcus uberis, Brucella melitensis, Corynebacterium bovis, Mycoplasma spp, Escherichia coli, Klebsiella pneumonia, Klebsiella oxytoca, Enterobacter aerogenes, Pasteurella spp, Trueperella pyogenes, Proteus spp, Prototheca zopfii and Prototheca wickerhamii.

In an embodiment, there is provided a method for predicting, diagnosing or monitoring endometritis in a cow, comprising

generating a glycosylation profile from a glycan-containing test sample of the cow,

determining from the glycosylation profile the degree of fucosylation of one or more glycans present in the test sample,

comparing the degree of fucosylation of the one or more glycans to one or more threshold values, and

providing a prediction or diagnosis of endometritis based on the comparison.

The glycan-containing test sample may be any suitable sample obtained from the subject which contains glycans. Suitable test samples include, but are not limited to, any suitable biological fluid, tissue or cell, or component thereof. Suitable biological fluids include, but are not limited to, whole blood, blood plasma, blood serum, urine, saliva, mucus, and milk, optionally blood serum or milk. Suitable biological tissues include tissue biopsies. The test sample may be obtained by any suitable technique known in the art.

Optionally, the test sample comprises glycans of one or more isolated glycoconjugates, optionally selected from one or more isolated glycoproteins, glycopeptides, peptidoglycans and glycolipids, or a combination thereof, further optionally one or more isolated glycoproteins.

Optionally, the one or more isolated glycoproteins is selected from one or more immunoglobulins, further optionally immunoglobulin G (IgG). Still further optionally, the test sample comprises glycans of ruminant or equine serum, optionally glycans of bovine serum IgG or equine serum IgG, preferably glycans of bovine serum IgG.

Further optionally, the test sample may comprise glycans from whole serum. Thus, suitable glycan-containing test samples include the total protein N-glycome from serum, optionally from ruminant serum or equine serum, further optionally from bovine serum.

Still further optionally, the glycan-containing test sample may comprise glycans of acute phase proteins or any other glycosylated serum protein, optionally from ruminant serum or equine serum, further optionally from bovine serum.

Optionally, the method may comprise determining a plurality of test values of glycosylation markers of the infection or condition. For example, without intending to limit the invention thereto, the method may comprise determining a plurality of degrees of fucosylation of glycans and/or a plurality of degrees of contribution of glycans, in order to achieve a combined result, wherein the glycans being examined for each test may be the same or different.

Advantages of the invention include the following but are not limited thereto. The methods of the invention advantageously enable postpartum infections or conditions to not only be diagnosed, but also to be predicted. Therefore, it is possible either shortly after calving, or even before calving (prepartum), to predict whether the animal being tested is likely to develop the infection or condition. Once a condition such as endometritis or metritis is established in an animal, the ill effects, including reduced fertility, are immediate. Thus, the ability to predict which animals will develop such infections/conditions is very significant, as it allows for prophylactic intervention and treatment, thereby preventing the ill effects and reduced fertility associated with such infections/conditions. Furthermore, it is conveniently possible to continue to monitor such infections, using the methods of the invention.

### DESCRIPTION OF THE FIGURES

Embodiments of the present invention will now be described, with reference to the accompanying, non-limiting example and drawings, in which:

FIG. 1 shows a representative bovine IgG N-glycome chromatogram from pooled bovine serum that shows a typical profile from healthy (clean) subjects, indicating the features that are normally visible. Bovine IgG N-glycans were prepared on the automated HTP glycomics workstation and analysed by HILIC to prepare the chromatogram (described in the Example below). Each peak represents one or more specific glycans, as indicated in Table 1.

FIG. 2 shows representative chromatograms of clean (top) and endometritic cows (bottom) on day 7 postpartum. In the bottom chromatogram, representing an endometritic cow, the afucosylated glycans are significantly reduced. This concurs with the finding that the degree of fucosylation is increased in endometritic cows (see Results in the Example).

FIGS. 3, 4 and 5 show boxplots showing glycosylation differences between clean cows and endometritic cows prepartum (FIG. 5), on day 7 postpartum (FIG. 4), and on day 14 postpartum (FIG. 3). These boxplots were generated from analysing samples of 50 of the 97 animals tested in the Example (25 clean animals, 25 endometritic animals). Shown are the glycan percentage areas for individual peaks that were significantly changed in endometritis, i.e. peaks 3 (GP03), 10 (GP10), 11 (GP11), 12 (GP12) and 16 (GP16), as well as the combination of glycans 3, 10, 11, 12, 16 (referred to as ‘Combined’). Also shown are the degrees of fucosylation for the agalactosylated glycan A2G0 (Fuc ratio G0), the mono-galactosylated glycans A2[3]G1 (Fuc ratio G1a) and A2[6]G1 (Fuc ratio G1b), the bis-galactosylated glycan A2G2 (Fuc ratio G2), as well as the degree of fucosylation for all of these glycans (Fuc ratio all). For each of FIGS. 3, 4 and 5, viewing the drawings straight on, the “clean” results are shown on the left hand side of each boxplot, and the “endometritic” results are shown on the right hand side.

FIG. 6 shows line graphs indicating mean±SEM relative peak percentage area of glycan peaks 3, 10, 11, 12 and 16 for animals classed as clean (Δ), metritis (□) or endometritis (◯) on days −10, 7, 14, 21 and 50 relative to calving (where calving=day 0). These line graphs were generated from analysing samples from all 97 animals, as described in the Example, and are another way of representing the data shown in FIGS. 3, 4 and 5. Note: y-axis values have been modified to highlight glycan patterns. Y-axis: Relative Peak Percentage Area. X-axis: Day relative to calving.

### EXAMPLE

Materials and Methods

Animals and Management

Three Irish dairy herds (97 female pregnant cows) were recruited to the present study. After calving, all cows were housed as a single group in a free stall house with concrete flooring. The cubicle to cow ratio was ≧1:1 at all times, thus meeting the recommendations of the Farm Animal Welfare Council (FAWC, 1997). All procedures were carried out in Ireland in compliance with the Cruelty to Animals Act 1876, and experimental protocols were approved by the local Ethical Review Committee.

Uterine Health Assessment

Uterine health was assessed by vaginal mucus assessment (Clinical evaluation of postpartum vaginal mucus reflects uterine bacterial infection and the immune response in cattle; Williams E J, Fischer D P, Pfeiffer D U, England G C, Noakes D E, Dobson H, Sheldon I M. Theriogenology. 2005 Jan. 1; 63(1):102-17). Briefly, the vulva was thoroughly cleaned with a dry paper towel and a clean, lubricated, gloved hand was then inserted through the vulva. In each cow, the vagina and the external cervical ostium were palpated, and the mucus contents of the vagina withdrawn manually for examination. The vaginal mucus was assessed for colour, proportion and volume of pus, and a character score assigned as follows: 0=clear or translucent mucus; 1=mucus containing flecks of white or off-white pus; 2=<50 ml exudate containing ≧50% white or off-white mucopurulent material; and 3=>50 ml exudate containing purulent material, usually white or yellow, but occasionally sanguineous. A smell score was assigned as 0 for no smell, and 3 if a fetid odour was present.

In addition to mucus scoring, endometrial samples for cytologic examination were collected using a cytobrush (Minitub, Germany) on day 50 postpartum. This method involves brushing cells from the endometrium and after staining uterine infection is defined where the proportion of white blood cells in the sample exceeded 5%. To identify systemic signs of infection, rectal temperature was routinely measured and animals were monitored for behavioural changes.

Cows were classified into disease categories based on the definitions by Sheldon et al (2006) (Sheldon I M, Lewis G, LeBlanc S and Gilbert R. (2006). Defining postpartum uterine disease in cattle. Theriogenology, 65(2006), 1516-1530). Thus, animals were classed as being healthy, i.e. ‘clean’ if they had a mucus score of 0 or 1 on all timepoints, including days 14, 21 and 50 postpartum, and animals were classed as having the condition metritis if they had a mucus score of greater than 2 on day 7 and/or 14, followed by a mucus score of 0 or 1 from day 21 onwards. Endometritis is defined as infection or inflammation that occurs after 21 days postpartum. Thus, a diagnosis of the condition endometritis was made if a cow had a mucus score of greater than 2 on either day 21 or 50 postpartum.

Blood Sample Collection

A pre-partum blood sample was collected on a date estimated to be 7 days before calving; collection time averaged 10±1 days before calving with a range of −25 until −1 day prepartum. Further samples were collected on days 7, 14, 21 and 50 postpartum. It will be appreciated that the samples were taken as close to the actual day indicated as logistically possible; for “day 7”, collection time ranged from day 5 to day 11 postpartum, for “day 14”, collection time ranged from day 12 to day 18 postpartum, for “day 21”, collection time ranged from day 19 to day 25 postpartum. Blood was collected from the coccygeal vein or artery into plain evacuated tubes (BD Vacutainer Systems, Plymouth, UK) and transported to the laboratory. Following overnight incubation at 4° C., serum was separated by centrifugation at 4° C. for 10 min×1500 g and stored frozen at −20° C. until further glycomic analysis, which was conducted in batches of up to 96 as described below. All samples collected from the same cow at different timepoints, were analysed in the same batch.

Glycoprotein Affinity Purification

A 96-well IgG affinity purification plate (Thermo Scientific, 50 μL Protein G agarose resin per well) was pre-conditioned by washing three times with 500 μL binding buffer (0.1M sodium phosphate, 0.15 M NaCl, pH 7.4). Aliquots of serum (50 μL per sample) were diluted with an equal volume of binding buffer and filtered through a 96-well filter plate (Pall AcroPrep, 1 μm pore size glass fiber membrane) by centrifuge filtration. The filtrates were added to the Protein G plate, which was agitated on a robotic shaker at 700 rpm at room temperature for 30 min. Chinese hamster ovary cell culture IgG was isolated by first filtering the cell culture supernatants (400 μL per sample) through a 96-well filter plate (Pall AcroPrep, 1 μm pore size glass fiber membrane) and then transferring the filtrate to a pre-conditioned 96-well IgG affinity purification plate (Thermo Scientific, 50 μL Protein A agarose resin per well). Buffer was removed by vacuum filtration and the Protein G or Protein A resins were washed five times with washing buffer (500 μL per well, 0.1 M sodium phosphate, 0.15 M sodium chloride, 1% Triton-X, pH 7.4) with intermittent agitation at 700 rpm for 1 min at room temperature. Next, the resins were washed five times with binding buffer (500 μL per well) to remove residual detergent. A receiver plate was prepared by dispensing neutralization buffer (50 μL per well, 1 M Tris-hydrochloride, pH 9.0) into a 96-well 2 mL collection plate, which was placed into the robotic vacuum manifold. Elution buffer (200 μL per well, 0.2 M glycine-hydrochloride, pH 2.5) was added to the Protein G or Protein A resins followed by incubation at room temperature for 2 min without agitation. IgG was eluted by vacuum filtration and collected. This cycle was repeated once more and the collection plate was briefly centrifuged (4000 g, 1 min). 50% of the filtrate was transferred to a 96-well ultrafiltration plate (Pall Acroprep, Omega membrane, 10 kDa nominal molecular weight limit) and the solvent was removed by vacuum filtration (24 mm Hg, typically 30 min) or centrifuge filtration (3700 g, room temperature, typically 15 min). The flow-through was discarded, the remaining filtrate added to the ultrafiltration plate and solvent was removed by vacuum (24 mm Hg, typically 60 min) or centrifuge filtration (3700 g, room temperature, typically 30 min). The ultrafiltration plate was washed with water (200 μL per well).

Glycoprotein Denaturation and Glycan Release

Denaturation buffer (50 μL per well, 100 mM ammonium bicarbonate, 50 mM dithiothreitol, 0.1% sodium dodecyl sulfate) was dispensed into the ultrafiltration plate, which was placed on a robotic heater shaker and fully covered and insulated with an anti-evaporation lid. This assembly was incubated at 65° C. with agitation at 700 rpm for 20 min. After cooling to room temperature, an iodoacetamide solution (100 mM, 10 μL per well) was added, the ultrafiltration plate was covered with an anti-evaporation lid and incubated at room temperature with agitation at 700 rpm for 30 min. Excess reagents together with solvent were removed by vacuum (24 mm Hg, typically 30 min) or centrifuge filtration (3700 g, 15 min, room temperature) and the samples were washed with water (20 μL per well). Water (40 μL per well) and PNGase F (Prozyme R Glyco N-Glycanase R , code GKE-5006D, 10 μL per well, 0.5 mU in 1 M ammonium bicarbonate, pH 8.0) were sequentially added and the ultrafiltration plate was insulated with an anti-evaporation lid and incubated at 40 fiC with agitation at 700 rpm for 2 h. During this time, the Protein G or Protein A resin was regenerated by washing 6 times with regeneration buffer (500 μL per well, 0.2 M glycine/HCl, 0.5% Triton-X, pH 2.5), 5 times with elution buffer (500 μL per well) and finally 4 times with binding buffer (500 μL per well). Glycans were recovered from the ultrafiltration plate by centrifuge filtration (3700 g, 15 min, room temperature). The ultrafiltration membranes were washed with water (20 μL per well) and the filtrate was combined with the first filtrate to obtain a final volume of 50-60 μL per sample.

Hydrazide-Mediated Glycan Cleanup

Each well of a 96-well chemically inert filter plate (Millipore Solvinert, hydrophobic polytetrafluoroethylene membrane, 0.45 μm pore size) was washed with 100 μL methanol (MeOH). Ultralink hydrazide resin (40 μL of a suspension in water, Thermo Scientific) was dispensed to each well. The resin was sequentially washed with MeOH, H₂O and acetonitrile (MeCN) and the plate was placed on a heater (70 MeCN/acetic acid (98:2) was added to the resin, followed by 20 μL of the glycan solution). The filter plate was incubated with shaking at 700 rpm at 70° C. for 45 min. 50 μL MeCN/acetic acid (98:2) were added and shaking was continued at the same temperature for 10 min to disrupt resin aggregates. The resin was washed sequentially with MeOH, guanidine, H₂O, triethylamine/MeOH (1:99) and MeOH (200 μL per well). Fresh MeOH (180 μL) and acetic anhydride (20 μL) were added and the plate was incubated for 10 min with agitation at 700 rpm. Excess reagent was removed by filtration and the resins were washed sequentially with MeOH, H₂O and MeCN. Acetic acid/MeCN (2:98, 180 μL) and H₂O (20 μL) were sequentially added and the plate was incubated at 70° C. with agitation at 700 rpm for 60 min. Fluorescent labelling mix (50 μL, 350 mM 2-aminobenzamide (2-AB), 1 M sodium cyanoborohydride in acetic acid/dimethyl sulfoxide (30:70)) was dispensed into each well and the plate was incubated at 70° C. with agitation at 700 rpm for 120 min.

Glycan Solid Phase Extraction

The labelling reaction was quenched by the addition of 200 μL MeCN/H₂O (95:5). The suspension was transferred to a 2 mL collection plate containing 800 μL MeCN/H₂O (95:5) per well, the beads were left to settle, 200 μL of the supernatant was aspirated and dispensed back into the filter plate. After extensive mixing the suspension was transferred back into the collection plate. This cycle was repeated once more to ensure a quantitative transfer of the resins. HyperSep Diol SPE cartridges (Thermo Scientific) were washed with 1 mL MeCN/H₂O (95:5), 1 mL H₂O and 1 mL MeCN/H₂O. Next, the beads were suspended and transferred onto the SPE cartridges. A 10 min incubation typically led to complete drainage of the solvent by gravity. The SPE cartridges were washed three times with 700 μL MeCN/H₂O (95:5). A collection plate was placed inside the robotic vacuum manifold and the SPE cartridges were washed twice with 200 μL H₂O/MeCN (80:20), with an intermittent incubation period of 10 min. The samples were concentrated to dryness in a vacuum evaporator (typically 4-6 h for 96 samples). The samples were dissolved in 30 μL MeCN/H₂O (70:30) and filtered (Pall Acroprep GHP membrane, 0.45 μm pore size). A 10 μL aliquot of the filtrate was analyzed by UPLC.

Ultra Performance Liquid Chromatography (UPLC)

2-aminobenzamide (2-AB) derivatized N-glycans were separated by UPLC with fluorescence detection on a Waters Acquity UPLC H-Class instrument consisting of a binary solvent manager, sample manager and fluorescence detector under the control of Empower 3 chromatography workstation software (Waters, 34 Maple Street, Milford, Mass. 01757 USA). The hydrophilic interaction liquid chromatography (HILIC) separations were performed using a Waters Ethylene Bridged Hybrid (BEH) Glycan column, 150×2.1 mm i.d., 1.7 μm BEH particles, with 50 mM ammonium formate, pH 4.4, as solvent A and MeCN as solvent B. The separation was performed using a linear gradient of 70-53% MeCN at 0.56 mL/min in 16.5 min for IgG separation. An injection volume of 10 μL sample prepared in 70% v/v MeCN was used throughout. Samples were maintained at 5° C. prior to injection and the separation temperature was 40° C. The fluorescence detection excitation/emission wavelengths were λex=330 nm and λem=420 nm, respectively. The system was calibrated using an external standard of hydrolyzed and 2AB-labeled glucose oligomers to create a dextran ladder, as described previously (Royle et al. Anal Biochem. 2008, 376(1):1-12). Empower software, referred to above, was used to process chromatography data. A fifth-order polynomial distribution curve was fitted to the dextran ladder to assign glucose unit (GU) values from retention times.

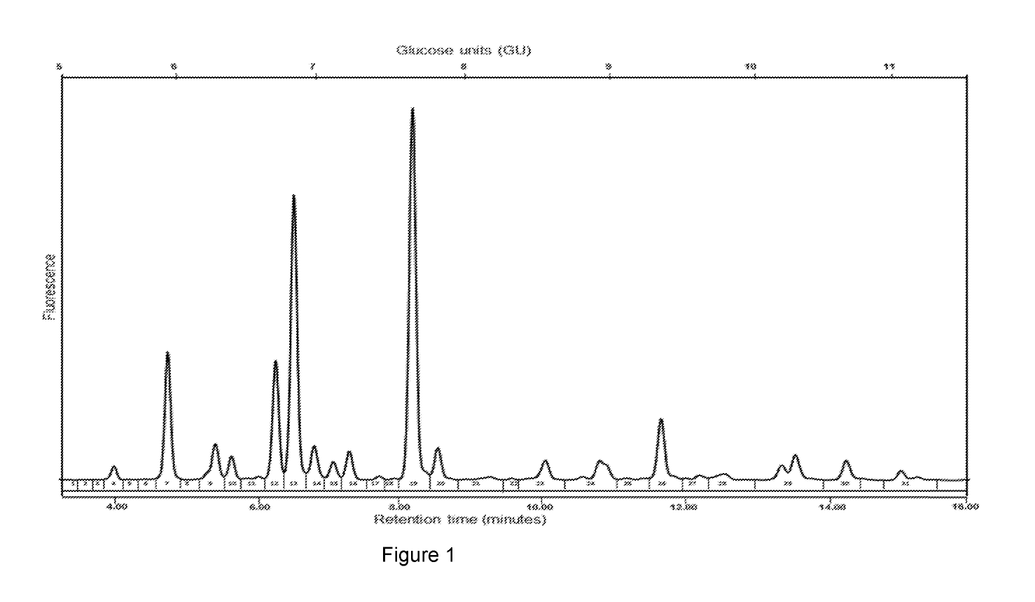

All of the samples were analysed as described above. A representative bovine IgG N-glycome chromatogram was generated from pooled bovine serum (FIG. 1). A chromatogram was generated for each sample and analysed against the FIG. 1 chromatogram, following which glycosylation markers (e.g. glycan peak areas) were examined. The following statistical analysis was then carried out on the values of the glycosylation markers obtained from clean and endometritic animals (50 of the 97 animals: 25 clean, 25 endometritic).

Statistical Analysis

Multiple analysis of variance (MANOVA) was performed to investigate the main and interaction effects of categorical variables (disease phenotype, time point, farm) on the glycan percentage areas obtained from clean and endometritis samples (50 of the 97 animals: 25 clean, 25 endometritic) using the R software package, available from The R Foundation for Statistical Computing (http://www.r-project.org). Tukey's HSD (‘Honestly significant difference’) test was performed to calculate p-values. The add-on software ggplot, available from http://ggplot2.org was used to generate boxplots. Receiver Operating Characteristics (ROC) analyses were performed using the pROC add-on software, available from web.expasy.org/pROC as described in Xavier Robin et al (2011). pROC: an open-source package for R and S+ to analyze and compare ROC curves. BMC Bioinformatics, 12, p. 77. DOI: 10.1186/1471-2105-12-77, to create ROC curves and calculate the area under the curve (AUC) (a C-statistic), sensitivity, specificity and accuracy. The optimal thresholds, which may be used as reference values for future test samples, were calculated using Youden's method as described in W. J. Youden (1950) “Index for rating diagnostic tests”. Cancer, 3, 32-35, maximising the difference between the true positive rate (PPV) and the false positive rate (NPV). Thus, Youden's method conveniently maximises specificity and sensitivity.

Results

A total of 97 animals were included in the study.

Disease Incidence

Clean—26.8% (26/97)

Metritis—47.4% (46/97)

Endometritis—25.8% (25/97)

Glycan Identification and Quantitation

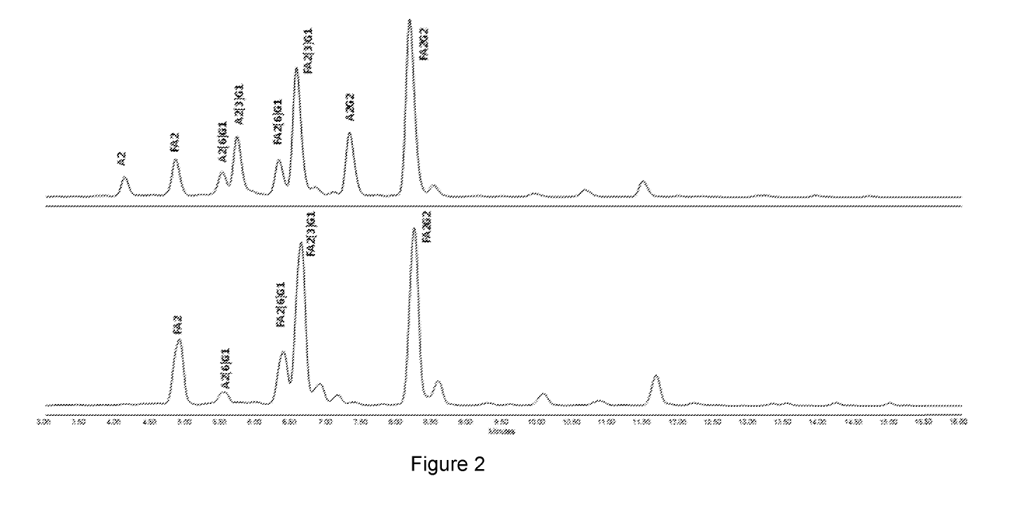

IgG N-glycans were isolated from bovine serum and fluorescently labelled using the automated HTP glycomics workstation as described above. Representative chromatograms are shown in FIGS. 1 and 2, as described above. Referring to FIG. 1, using HILIC with fluorescence detection, the glycans were separated into 31 glycan peaks (shorthand ‘GP’), each peak representing a specific glycan or a mixture of glycans, as shown in Table 1 below.

Monosaccharide Symbol

| | | |
|---|---|---|
| ▪ | N-acetylglucosamine | GlcNAc |
| □ | Glucose | Glc |
| ⋄ | Galactose | Gal |
| ♦ | N-acetylgalactosamine | GalNAc |
| [symbol] | Fucose | Fuc |
| ○ | Mannose | Man |
| ★ | N-acetylneuraminic acid | NeuNAc |
| Δ | Xylose | Xyl |

Linkage Postion

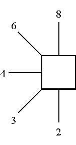

Linkage Type

| | |
|---|---|
| - - - | α linkage |
| — | β linkage |
| [symbol] | unknown α linkage |
| [symbol] | unknown β linkage |

TABLE 1

| | | Average | | |
|---|---|---|---|---|
| | | percentage | | |
| Peak | GU | area | Structure | UOXF Symbol |
| GP3 | 5.20 | 0.23 | likely A1 | likely 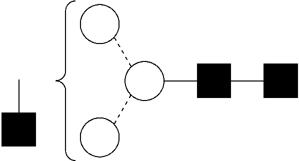 |
| GP4 | 5.37 | 1.38 | A2G0 | 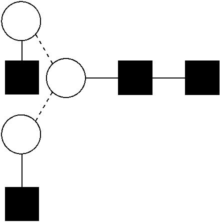 |
| GP5 | 5.54 | 0.2 | A2B | 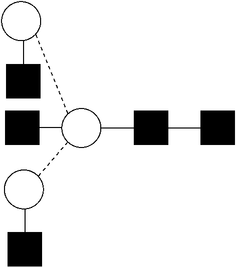 |
| GP6 | 5.64 | 0.29 | A1G1 | 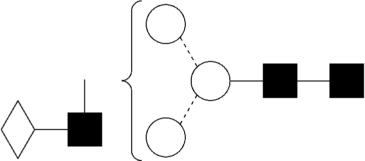 |
| GP7 | 5.82 | 9.49 | FA2G0 | 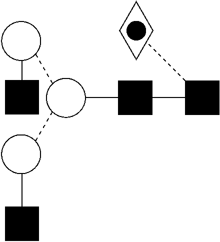 |
| GP8 | 6.08 | 0.27 | FA2B | 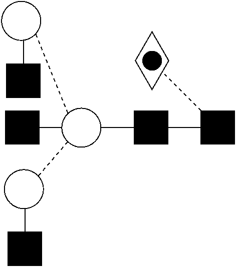 |
| GP9 | 6.18 | 3.95 | A2[6]G1 | 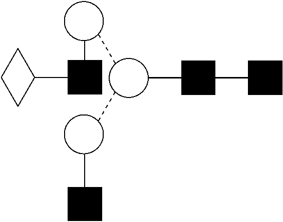 |
| GP10 | 6.29 | 2.28 | A2[3]G1 | 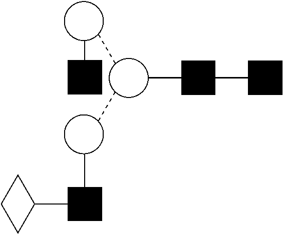 |
| GP11 | 6.45 | 0.88 | A2B[6]G1 | 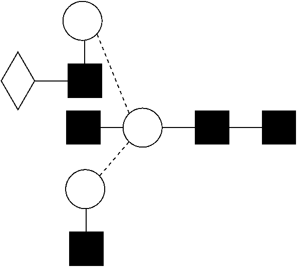 |
| | | | A2B[3]G1 | 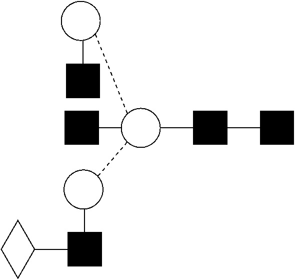 |
| GP12 | 6.6 | 7.74 | FA2[6]G1 | 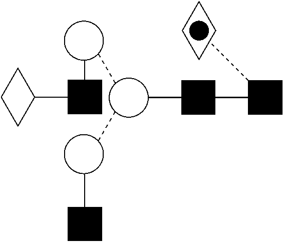 |
| GP13 | 6.72 | 23.8 | FA2[3]G1 | 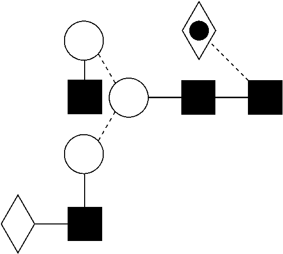 |
| GP14 | 6.86 | 3.16 | FA2B[6]G1 | 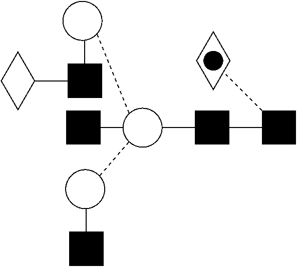 |
| GP15 | 6.98 | 1.27 | FA2B[3]G1 | 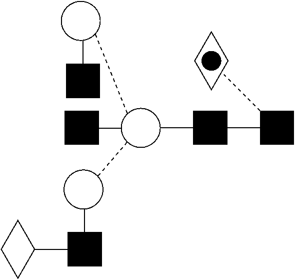 |
| GP16 | 7.09 | 2.84 | A2G2 | 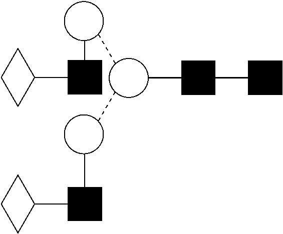 |
| GP17, GP18 | 7.29 | 0.37 | A2BG2 | 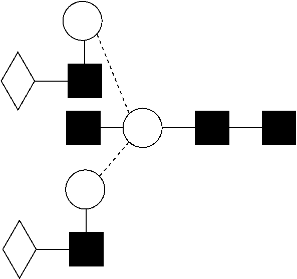 |
| GP19 | 7.51 | 27.04 | FA2G2 | 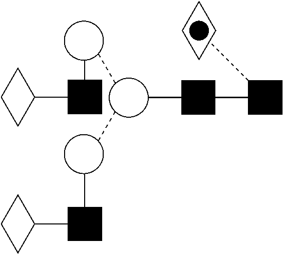 |
| GP20 | 7.67 | 2.91 | FA2BG2 | 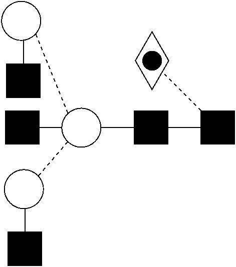 |
| | | | FA1G1S1 | 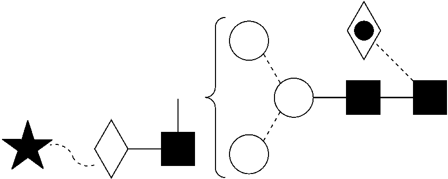 |
| GP21 | 8.02 | 0.35 | A2[6]G1S1 | 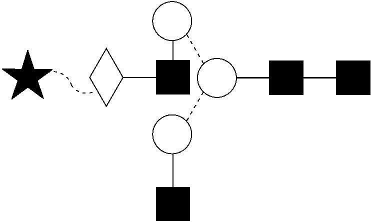 |
| | | | A2[3]G1S1 | 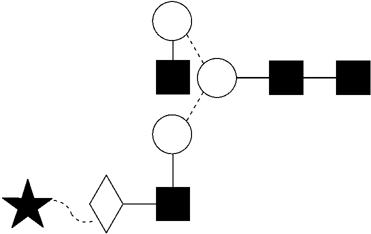 |
| GP22 | 8.17 | 0.22 | A2B[6]G1S1 | 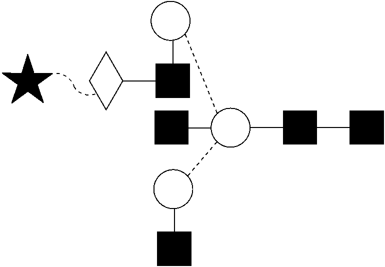 |
| | | | A2B[3]G1S1 | 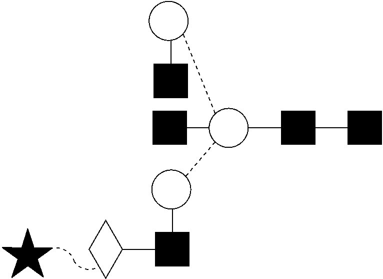 |
| GP23 | 8.4 | 1.41 | FA2G2Sg1 | 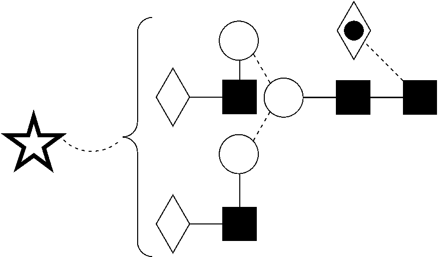 |
| | | | A2G2Sg1 | 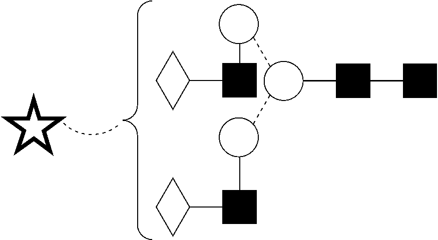 |
| GP24 | 8.8 | 1.57 | FA2G2S1(a2, 6) | 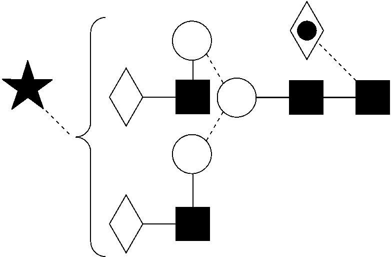 |
| | | | A2G2S1 | 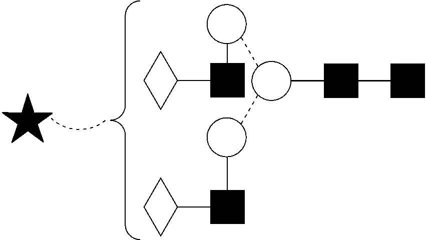 |
| GP25 | 8.96 | 0.25 | FA2BG2S1 | 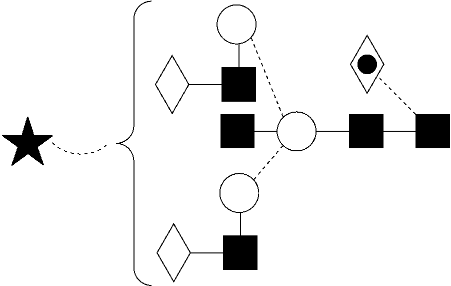 |
| GP26 | 9.21 | 3.95 | FA2G2Sg1 | 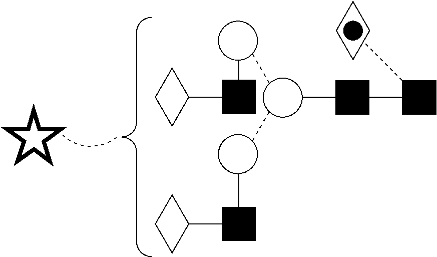 |
| GP27 | 9.46 | 0.51 | FA2BG2Sg1 | 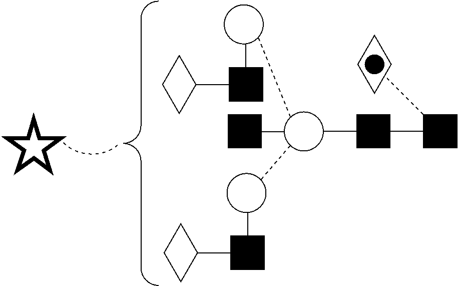 |
| GP28 | 9.63 | 0.41 | A2G1S1 | 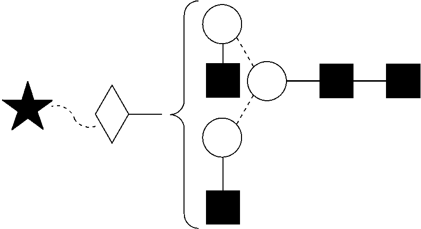 |
| | | | A2G2S2 | 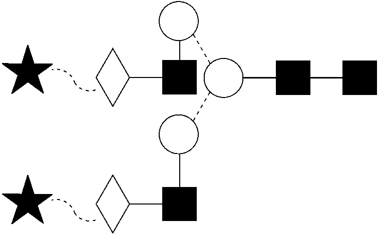 |
| GP29 | 10.01- 10.13 | 0.58 | A2G2Sg2 | 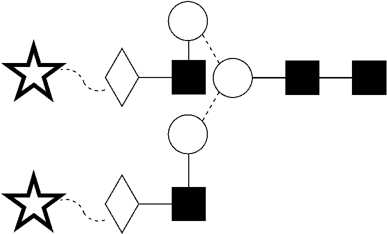 |
| | | 1.2 | FA2G2Sg2 | 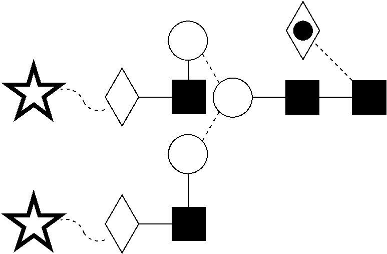 |
| | | | FA2BG2Sg2 | 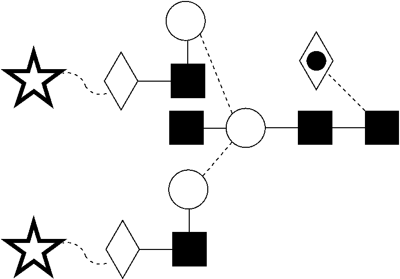 |
| GP30 | 10.48 | 0.97 | A2G2Sg2 | 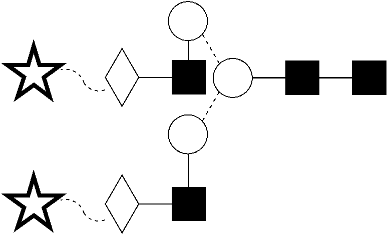 |
| | | | FA2G2S1Sg1 | 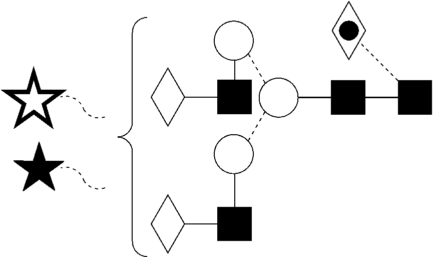 |
| | | | FA2G2Sg2 | 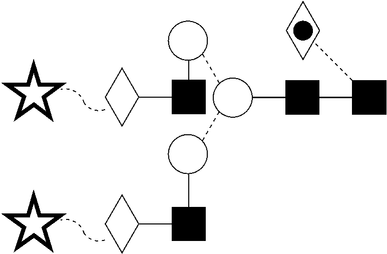 |
| GP31 | 10.91 | 0.72 | FA2BG2Sg2 | 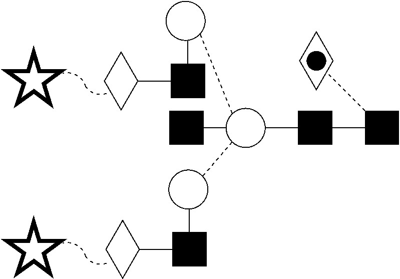 |

In Table 1, the UOXF Symbol is the standard Oxford notation, as described in e.g. Pauline M. Rudd et al, Proposal for a standard system for drawing structural diagrams of N- and O-linked carbohydrates and related compo, Proteomics 2009, 9, 3796-3801. Thus, for example, A2[6]G1 is a mono-galactosylated glycan comprising a galactose linked to the 6-arm of the structure A2, and FA2[6]G1 is the fucosylated version of A2[6]G1.

Some peaks were found to co-elute so that one peak was representative of two or more glycans. For instance, GP11 contained the two structural isomers of A2BG1. The relevant peaks which are most discriminatory contain only one glycan, with the exception of peak 11 (GP11) which as a whole has discriminatory power. It will be appreciated by a skilled person that the co-elution of some peaks does not affect the results generated and reported below, including the threshold values. Thus, the threshold values discussed below can conveniently be used to predict or diagnose endometritis in a cow.

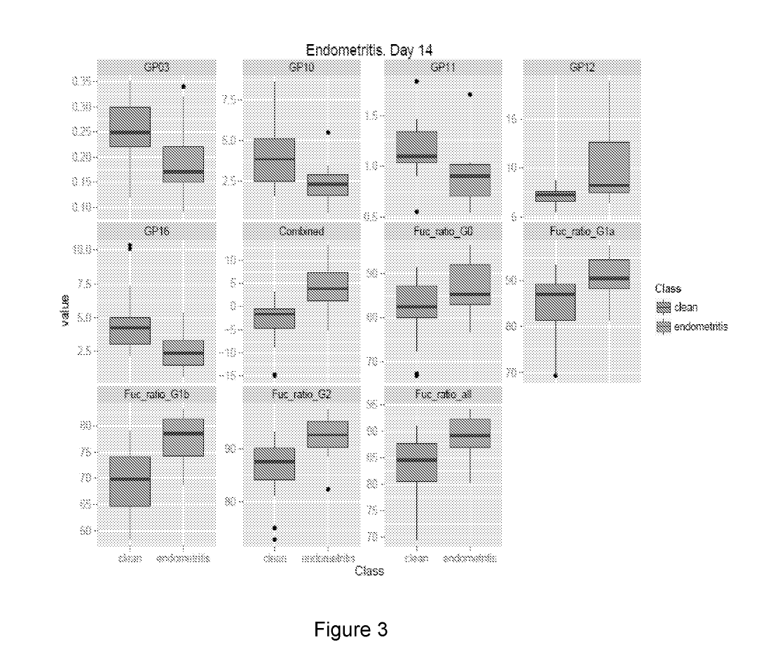

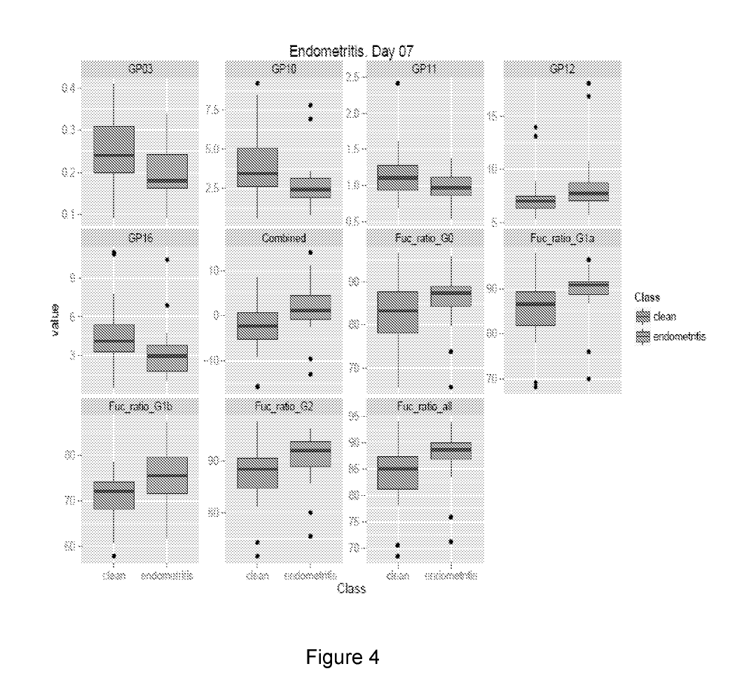

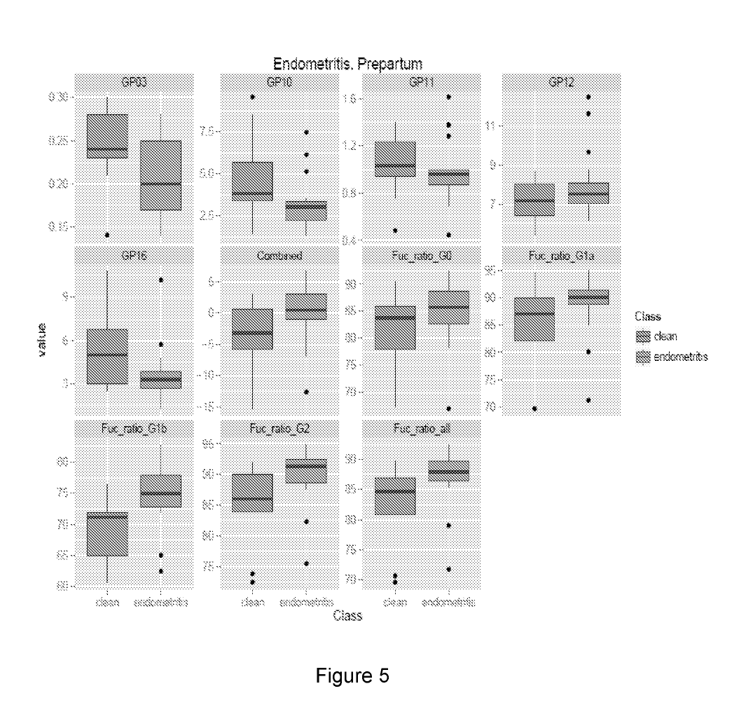

As mentioned above, the chromatograms generated for each sample were analysed against the FIG. 1 chromatogram. The samples from 50 animals of the 97 animals tested (25 clean, 25 endometritic) were analysed further as follows. The peak areas, which are proportional to the glycan quantities, were measured and expressed as glycan percentage areas. In addition, the degrees of fucosylation (referred to as fucosylation ratios, or the fucosylation index) were calculated by dividing the peak area of each fucosylated glycan by the sum of peak areas of the fucosylated and afucosylated glycan. Boxplots were generated to illustrate these data, e.g. as shown in FIGS. 3, 4 and 5. The degree of fucosylation was determined for the agalactosylated glycan A2G0 (‘Fuc ratio G0’), the mono-galactosylated glycans A2[3]G1 (‘Fuc ratio G1a’) and A2[6]G1 (‘Fuc ratio G1b’), the bis-galactosylated glycan A2G2 (‘Fuc ratio G2’) as well as for the combination of these glycans (‘Fuc ratio all’). Multivariate statistical analysis, described above, was carried out on the data represented in the boxplots. Receiver operator characteristic (ROC) curves were constructed for the glycan peaks which were identified as being significantly changed in endometritis to assess their capability of distinguishing endometritic animals from clean ones. A ROC curve shows the performance of a binary classification method and shows the sensitivity (the proportion of correctly classified positive cases) and specificity (the proportion of correctly classified negative cases) as the output threshold is moved over the range of all possible values. The results are shown in Table 2.

TABLE 2
Receiver operating characteristic (ROC) analysis data and p-values for glycan Peaks
3, 10, 11, 12, 16 and combined, and for the degrees of fucosylation of the glycans
A2G0, A2[3]G1, A2[6]G1 and A2G2 and combined, for subjects prepartum and
on days 7 and 14 postpartum. Thresholds were determined with Youden's index.

| | | | | | | | | | | | | |
|---|---|---|---|---|---|---|---|---|---|---|---|---|
| | Thresh- | Speci- | Sensi- | Accu- | | | | | | | | p- |
| Feature | old | ficity | tivity | racy | TN | TP | FN | FP | NPV | PPV | AUC | value |

Prepartum

| | | | | | | | | | | | | |
|---|---|---|---|---|---|---|---|---|---|---|---|---|
| Fuc index A2G0 | 78.13 | 33.33 | 94.12 | 0.73 | 3 | 16 | 1 | 6 | 75.0 | 72.7 | 60.8 | 0.24 |
| Fuc index A2[3]G1 | 88.24 | 66.67 | 76.47 | 0.73 | 6 | 13 | 4 | 3 | 60.0 | 81.3 | 66.0 | 0.14 |
| Fuc index A2[6]G1 | 72.00 | 77.78 | 82.35 | 0.81 | 7 | 14 | 3 | 2 | 70.0 | 87.5 | 81.6 | 0.01 |
| Fuc index A2G2 | 86.84 | 55.56 | 88.24 | 0.77 | 5 | 15 | 2 | 4 | 71.4 | 78.9 | 72.2 | 0.04 |
| Fuc index combined | 85.64 | 66.67 | 82.35 | 0.77 | 6 | 14 | 3 | 3 | 66.7 | 82.4 | 74.1 | 0.06 |
| GP03 | 0.21 | 88.89 | 64.71 | 0.73 | 8 | 11 | 6 | 1 | 57.1 | 91.7 | 72.4 | 0.07 |
| GP10 | 3.41 | 77.78 | 76.47 | 0.77 | 7 | 13 | 4 | 2 | 63.6 | 86.7 | 69.2 | 0.10 |
| GP11 | 0.99 | 66.67 | 70.59 | 0.69 | 6 | 12 | 5 | 3 | 54.5 | 80.0 | 58.9 | 0.70 |
| GP12 | 7.19 | 55.56 | 70.59 | 0.65 | 5 | 12 | 5 | 4 | 50.0 | 75.0 | 57.3 | 0.25 |
| GP16 | 4.92 | 55.56 | 88.24 | 0.77 | 5 | 15 | 2 | 4 | 71.4 | 78.9 | 66.4 | 0.08 |
| GP3, 10, 11, 12, 16 | −2.38 | 55.56 | 82.35 | 0.73 | 5 | 14 | 3 | 4 | 62.5 | 77.8 | 67.6 | 0.07 |
| combined | | | | | | | | | | | | |

Day 7 Postpartum

| | | | | | | | | | | | | |
|---|---|---|---|---|---|---|---|---|---|---|---|---|
| Fuc index A2G0 | 85.97 | 70.00 | 66.67 | 0.68 | 14 | 12 | 6 | 6 | 70.0 | 66.7 | 64.6 | 0.16 |
| Fuc index A2[3]G1 | 88.56 | 75.00 | 77.78 | 0.76 | 15 | 14 | 4 | 5 | 78.9 | 73.7 | 69.3 | 0.09 |
| Fuc index A2[6]G1 | 75.16 | 90.00 | 61.11 | 0.76 | 18 | 11 | 7 | 2 | 72.0 | 84.6 | 71.7 | 0.03 |
| Fuc index A2G2 | 90.40 | 75.00 | 72.22 | 0.74 | 15 | 13 | 5 | 5 | 75.0 | 72.2 | 69.3 | 0.09 |
| Fuc index combined | 86.33 | 70.00 | 77.78 | 0.74 | 14 | 14 | 4 | 6 | 77.8 | 70.0 | 70.3 | 0.08 |
| GP03 | 0.23 | 70.00 | 72.22 | 0.71 | 14 | 13 | 5 | 6 | 73.7 | 68.4 | 66.0 | 0.06 |
| GP10 | 3.36 | 60.00 | 83.33 | 0.71 | 12 | 15 | 3 | 8 | 80.0 | 65.2 | 65.8 | 0.10 |
| GP11 | 0.93 | 90.00 | 44.44 | 0.68 | 18 | 8 | 10 | 2 | 64.3 | 80.0 | 69.1 | 0.03 |
| GP12 | 7.37 | 75.00 | 66.67 | 0.71 | 15 | 12 | 6 | 5 | 71.4 | 70.6 | 68.1 | 0.16 |
| GP16 | 3.29 | 75.00 | 72.22 | 0.74 | 15 | 13 | 5 | 5 | 75.0 | 72.2 | 70.1 | 0.10 |
| GP3, 10, 11, 12, 16 | −1.41 | 65.00 | 77.78 | 0.71 | 13 | 14 | 4 | 7 | 76.5 | 66.7 | 68.3 | 0.05 |
| combined | | | | | | | | | | | | |

Day 14 Postpartum

| | | | | | | | | | | | | |
|---|---|---|---|---|---|---|---|---|---|---|---|---|
| Fuc index A2G0 | 81.28 | 42.86 | 92.86 | 0.63 | 9 | 13 | 1 | 12 | 90.0 | 52.0 | 67.5 | 0.05 |
| Fuc index A2[3]G1 | 87.68 | 61.90 | 92.86 | 0.74 | 13 | 13 | 1 | 8 | 92.9 | 61.9 | 74.6 | 0.01 |
| Fuc index A2[6]G1 | 75.05 | 80.95 | 71.43 | 0.77 | 17 | 10 | 4 | 4 | 81.0 | 71.4 | 82.2 | 0.00 |
| Fuc index A2G2 | 90.26 | 76.19 | 78.57 | 0.77 | 16 | 11 | 3 | 5 | 84.2 | 68.8 | 80.3 | 0.00 |
| Fuc index combined | 85.72 | 61.90 | 92.86 | 0.74 | 13 | 13 | 1 | 8 | 92.9 | 61.9 | 77.5 | 0.00 |
| GP03 | 0.21 | 85.71 | 71.43 | 0.80 | 18 | 10 | 4 | 3 | 81.8 | 76.9 | 72.7 | 0.01 |
| GP10 | 3.47 | 61.90 | 92.86 | 0.74 | 13 | 13 | 1 | 8 | 92.9 | 61.9 | 76.0 | 0.01 |
| GP11 | 1.05 | 71.43 | 92.86 | 0.80 | 15 | 13 | 1 | 6 | 93.8 | 68.4 | 77.8 | 0.01 |
| GP12 | 7.45 | 66.70 | 85.70 | 0.47 | 14 | 12 | 2 | 7 | 87.5 | 63.2 | 78.9 | 0.00 |
| GP16 | 2.12 | 100.00 | 50.00 | 0.80 | 21 | 7 | 7 | 0 | 75.0 | 100.0 | 78.5 | 0.00 |
| GP3, 10, 11, 12, 16 | 1.76 | 95.24 | 71.43 | 0.86 | 20 | 10 | 4 | 1 | 83.3 | 90.9 | 85.3 | 0.00 |
| combined | | | | | | | | | | | | |

TN: true negatives, TP: true positives, FN: false negatives, FP: false positives, NPV: negative predictive value, PPV: positive predictive value, AUC: area under the curve.

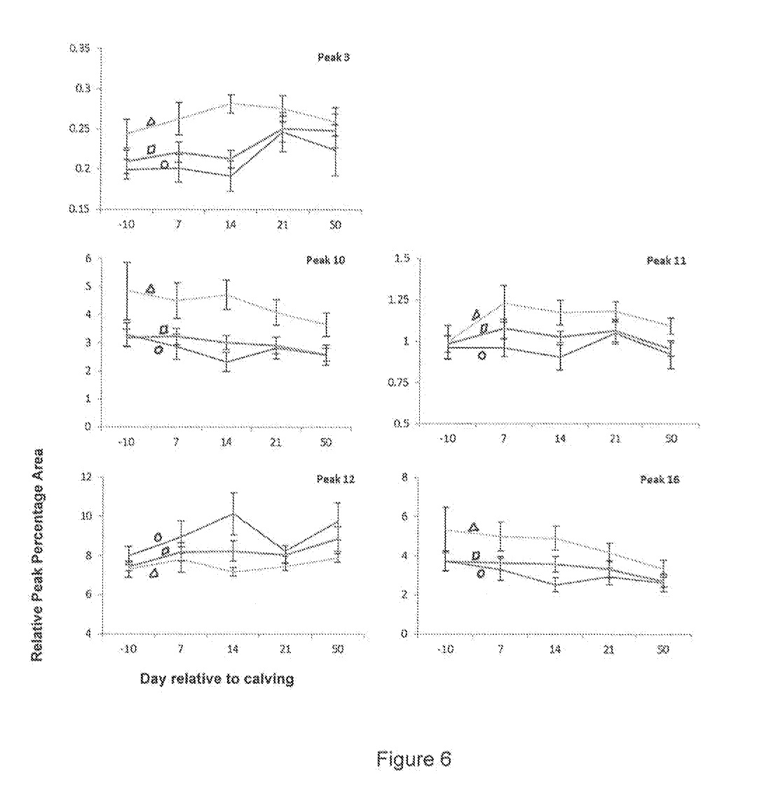

The statistical analysis carried out on the 25 clean and 25 endometritic animals showed that there was significant separation between cows with endometritis and control animals (p<0.01). For example, the relative percentage areas of Peaks 3, 10, 11 and 16 were significantly decreased (p<0.01) in the serum samples from endometritic cows when compared to clean cows on day 14 postpartum, and these peaks, identified in Table 2, contributed most to the separation of the clean and diseased groups. In particular, Peak 10 most significantly contributed to the separation of the groups. These peaks contain bi-antennary, asialylated glycans with zero, one and two galactose residues, with some having an alpha-1,6-linked (core) fucose residue. Specifically, peaks, 10, 11 and 16 represent, respectively, the glycans A2[3]G1, A2BG1 (mixture of isomers) and A2G2. Peak 12 was significantly increased (p<0.01) in the serum samples from endometritic animals. This peak contains the mono-galactosylated, core-fucosylated glycan FA2[6]G1. Based on these results, a glycan feature that combines the peak areas of Peaks 3, 10, 11, 12 and 16 was calculated by subtracting the sum of the peak areas of Peaks 3, 10, 11 and 16 from that of Peak 12. FIG. 6 is a further representation (by means of line graphs) which shows the mean±SEM relative percentage area of peaks 3, 10, 11, 12 and 16 for clean cows compared to cows with metritis and endometritis (i.e. all 97 animals) before calving and across the postpartum period.

The degrees of fucosylation of the glycans A2G0, A2[3]G1, A2[6]G1 and A2G2 were significantly increased (p<0.01) in the serum samples from endometritic animals compared to clean animals (Table 2). FIG. 2 shows representative chromatograms of clean (top) and endometritic cows (bottom) on day 7 postpartum, wherein, in the bottom chromatogram, the afucosylated glycans are significantly reduced which concurs with the finding that the degree of fucosylation is increased in endometritic cows. No significant changes in the degrees of galactosylation were observed. The fucosylation ratio of glycan A2[6]G1 (Fuc ratio G1b) gave the largest area under the curve (AUC) from the ROC analysis, with a sensitivity of 71% and specificity of 81% (Table 2).

Threshold values were calculated for significantly different glycan features and are also shown in Table 2. A P value of P≦0.05 represents a statistically significant result. The threshold values shown in Table 2 can conveniently be used to predict or diagnose endometritis in a cow. For example, on day 14 postpartum, animals with a degree of fucosylation (Fuc index) of the glycan A2[6]G1, above 75.05%, are more likely to be endometritic, and animals with a degree of fucosylation (Fuc index) of the glycan A2[6]G1, below 75.05%, are likely to be clean (P<0.05; median specificity 81%, median sensitivity 71%). Thus, a degree of fucosylation of the glycan A2[6]G1 present in a day 14 test sample, above a threshold value of about 75%, is indicative of a prediction of endometritis (which can be clinically confirmed from day 21 onwards). Furthermore, for example, for a prepartum sample, animals with a degree of fucosylation (Fuc index) of the glycan A2[6]G1, above 72.0%, are more likely to be endometritic, and animals with a degree of fucosylation (Fuc index) of the glycan A2[6]G1, below 72.0%, are likely to be clean (P<0.05; median specificity 78%, median sensitivity 82%). Thus, a degree of fucosylation of the glycan A2[6]G1 present in a prepartum test sample, above a threshold value of about 72%, is indicative of a prediction of endometritis (which can be clinically confirmed from day 21 onwards).

As another example using a prepartum sample, animals with a degree of fucosylation (Fuc index) of the glycan A2G2, above 86.8%, are more likely to be endometritic, and animals with a degree of fucosylation (Fuc index) of the glycan A2[6]G1, below 86.8%, are likely to be clean (P<0.05; median specificity 56%, median sensitivity 88%).

Furthermore, for example, on day 14 postpartum, animals with a degree of contribution of peak 10, below 3.47%, are more likely to be endometritic, and animals with a degree of contribution of peak 10, above 3.47%, are more likely to be clean (P<0.05; median specificity 62%, median sensitivity 93%).

It will be appreciated that the threshold value depends on a number of factors, including (i) the glycosylation marker; (ii) the glycan investigated; and (iii) the test day.

In summary, the method of the invention conveniently enables not just diagnosis of postpartum infections or conditions, but also enables an accurate prediction of whether or not the animal will develop a postpartum infection or condition such as endometritis.

1. A method for predicting, diagnosing or monitoring an infection or condition caused by or associated with one or more microorganisms in a non-human female animal subject, comprising
generating a glycosylation profile from a glycan-containing test sample of the subject,
determining from the glycosylation profile one or more test values of one or more glycosylation markers of the infection or condition,
comparing the one or more test values of the one or more glycosylation markers to one or more threshold values of the one or more glycosylation markers, and
providing a prediction or diagnosis of the infection or condition based on the comparison.

2. A method according to claim 1, wherein the one or more test values of the one or more glycosylation markers comprises one or more levels of fucosylation of one or more glycans present in the test sample.

3. A method according to claim 1, wherein the one or more test values of the one or more glycosylation markers comprises the degree of fucosylation of one or more glycans present in the test sample.

4. A method according to claim 2, wherein the one or more glycans is selected from the glycans A2G0, A2[3]G1, A2[6]G1 and A2G2, or a combination thereof.

5. A method according to claim 4, wherein a degree of fucosylation of the glycan A2[6]G1 present in a prepartum test sample, above a threshold value of about 72%, is indicative of a prediction of the infection or condition.

6. A method according to claim 4, wherein a degree of fucosylation of the glycan A2[6]G1 present in a day 7 test sample, above a threshold value of about 75%, is indicative of a prediction or diagnosis of the infection or condition.

7. A method according to claim 4, wherein a degree of fucosylation of the glycan A2[6]G1 present in a day 14 test sample, above a threshold value of about 75%, is indicative of a prediction or diagnosis of the infection or condition.

8. A method according to claim 1, wherein the one or more test values of the one or more glycosylation markers comprises the degree of contribution of one or more glycans present in the test sample to the total glycan pool.

9. A method according to claim 1, wherein the non-human female animal subject is a cow or a horse.

10. A method according to claim 9, wherein the non-human female animal subject is a cow.

11. A method according to claim 1, wherein the infection or condition caused by or associated with one or more microorganisms is selected from an infection or condition of the reproductive tract or a part thereof or an infection or condition of the mammary gland, or a combination thereof.

12. A method according to claim 1, wherein the infection or condition caused by or associated with one or more microorganisms is selected from metritis, endometritis and mastitis, or a combination thereof.

13. A method according to claim 12, wherein the infection or condition is endometritis.

14. A method according to claim 1, wherein the test sample is selected from whole blood, blood plasma, blood serum, urine, saliva, mucus, and milk.

15. A method according to claim 14, wherein the test sample is selected from blood serum or milk.

16. A method according to claim 1, wherein the test sample comprises glycans of one or more isolated glycoproteins.

17. A method according to claim 16, wherein the test sample comprises glycans of bovine serum immunoglobulin (IgG).

18. A method according to claim 2, wherein the one or more test values of the one or more glycosylation markers comprises the degree of fucosylation of one or more glycans present in the test sample.

19. A method according to claim 3, wherein the one or more glycans is selected from the glycans A2G0, A2[3]G1, A2[6]G1 and A2G2, or a combination thereof.
